(19) United States
(12) Patent Application Publication
(10) Pub. No.: US 2026/0053468 A1
(43) Pub. Date: Feb. 26, 2026

(54) SYSTEM AND METHOD FOR VERIFYING THAT WEARABLE ULTRASONIC SAMPLING DEVICE IS PROPERLY PLACED ON BODY

(71) Applicant: IKKO Health Ltd., Tel Aviv (IL)

(72) Inventors: Ramon Axelrod, Kiryat Ono (IL); Pinchas Chaviv, Hod HaSharon (IL); Michal Cohen Sfady, Ramat Gan (IL)

(73) Assignee: IKKO Health Ltd., Tel Aviv (IL)

(21) Appl. No.: 19/102,473

(22) PCT Filed: Aug. 22, 2023
(86) PCT No.: PCT/IB2023/058338

§ 371 Date: Feb. 10, 2025

Related U.S. Application Data
(60) Provisional application No. 63/400,758, filed on Aug. 25, 2022.

(51) Int. Cl. A61B 8/00

(52) U.S. Cl. CPC A61B 8/4427; A61B 8/4263; A61B 8/4472; A61B 8/4488; A61B 8/483

(57) ABSTRACT

The present invention provides information to professional staff performing an ultrasonic procedure either by using low resolution images, which can be produced rapidly in real time, or by evaluating the garment position based on initial reflection patterns on specific key points even without direct imaging of those specific key points.

### FIELD OF THE INVENTION

The present invention generally relates to ultrasonic imaging, and particularly to a system and a method for verifying that a wearable ultrasonic sampling device is properly placed on the patient's body.

### BACKGROUND OF THE INVENTION

Ultrasound (US) is one of the most widely available medical imaging modalities. Ultrasound is mainly used to image soft tissues and is considered safe. Current ultrasound systems consist of an ultrasonic pod or transducer that emits and receives ultrasound signals, and accompanying hardware that analyzes the received or reflected signals and generates an image. Medical ultrasound typically operates at frequencies between 3 MHz and 15 MHz, although 1 MHz also exists in older type equipment.

In the development of medical ultrasound, the ultrasonic transceiver has shifted from piezo-based emitters/receivers to silicon/MEMS (micro-electromechanical systems) based devices. This shift has enabled tighter integration between transceiver and electronics drivers, which in turn, has led to standard US equipment becoming smaller in size and cheaper in price. This has resulted in many small US devices for outpatient clinical use and, in some cases, home use. Classic US has a transceiver device that emits US energy and uses reflections and Time-of-Flight (ToF) analysis to generate a 2D image of internal organs.

An alternative method that has been used recently is US hardware that encircles the patient's body part (e.g., the patient's breast) to be scanned and which exploits reflections, refractions and transmission of US signals in order to gain much more information and build accurate 3D models of the patient's internal organs.

US processing is also used in geophysics, specifically in the oil & gas industry. The US processing involves recording ultrasonic stimulus (transmit) at multiple locations and the resulting reflections (receive), also at multiple, potentially at different locations, from underground layers to perform an inversion operation and estimate what could be the underground layers that caused the receive signals to form.

The basic mathematics and physics behind the inversion process has two parts:
1. Forward wave propagation: the transmit signals are propagated mathematically, by simulation, through an assumed model of the scanned medium.
2. Backward error propagation: the actually received signals are compared with the simulated receive signals and the errors are propagated “backwards” through the assumed model.

These two steps are repeated iteratively until the error between the actual and computed receive signals is lower than a predetermined level. At that point the assumed model reflects the underground layers and elements.

The entire process of getting to the underground structure model from the transmit and receive signals is called inversion.

In the case of medical imaging the transmitters and receivers are placed at random, or periodic, locations around or on part of the patient's body. US transmit signals are sent at one or several frequencies and the US sensors receive reflected, refracted and through transmitted signals. An inversion process, similar to the one described, follows and a 3D model of the internal organs of the patient is computed.

However, there is a major problem of using the inversion process for medical imaging: In the case of medical US imaging, there is a very large time disparity between the time it takes to sample data for an inversion process and the time it takes to perform the actual inversion and generate the internal 3D structure. The sampling process can take as little as several hundred milliseconds or very few seconds, but the inversion process might take hours or scores of minutes to complete, depending on the required resolution of the 3D model.

The length of time to complete an inversion process stands in stark contrast to a classic US imaging that is “real time”—a price paid for the much improved resolution.

Performing the inversion operation is highly computationally intensive. While an inversion at low resolution may take minutes to perform, one with high resolution may take hours, or even days to complete. In general the relation between resolution and compute time (and hence cost) is 1: X⁴. This extreme, exponentially rising, ratio is the problem tackled by the present invention.

### SUMMARY OF THE INVENTION

The present invention is applicable for use with a wearable ultrasonic sampling device (such as but not limited to, a garment, a belt and the like) that has ultrasonic sensors and which is placed on a patient's body to acquire ultrasonic images of body parts.

In order to avoid lengthy computation processes, the imaging system must verify that the wearable ultrasonic sampling device is properly placed or worn on the patient's body.

The present invention does not need high resolution to determine that the garment or other ultrasonic sampling device is misplaced, and does not need any image at all. Instead the present invention provides information to the professional staff performing the US procedure either by using low resolution images, which can be produced rapidly in real time, or by evaluating the garment position based on initial reflection patterns on specific key points even without direct imaging of those specific key points.

### BRIEF DESCRIPTION OF THE DRAWINGS

The present invention will be understood and appreciated more fully from the following detailed description, taken in conjunction with the drawings in which:

FIG. 1 is an illustration of an ultrasonic image produced by a small phased array which provides an immediate low resolution image of a specific area following placement of a wearable ultrasonic sampling device on the patient's body, in accordance with a non-limiting embodiment of the invention.

FIG. 2 is an illustration of an ultrasonic image produced by high frequency transceivers located on specific locations on a wearable ultrasonic sampling device on the patient's body, in accordance with a non-limiting embodiment of the invention.

FIG. 3 is an illustration of a method of evaluating patient scan quality by a wearable ultrasound device, in accordance with a non-limiting embodiment of the invention.

FIG. 4 is an illustration of a method of evaluating patient scan quality by a wearable ultrasound device, in accordance with another non-limiting embodiment of the invention.

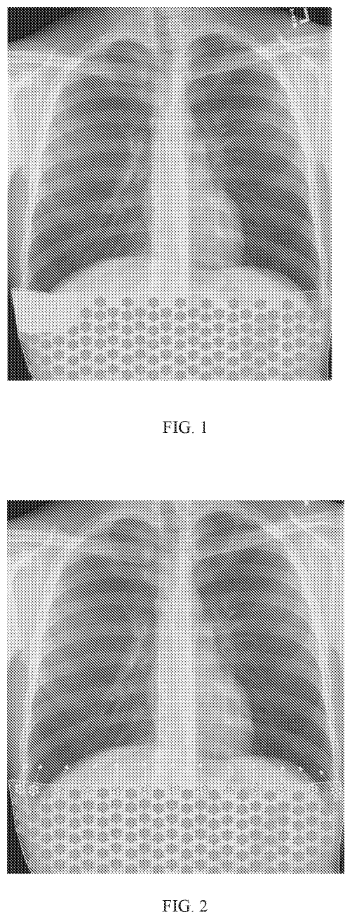

### DETAILED DESCRIPTION OF EMBODIMENTS

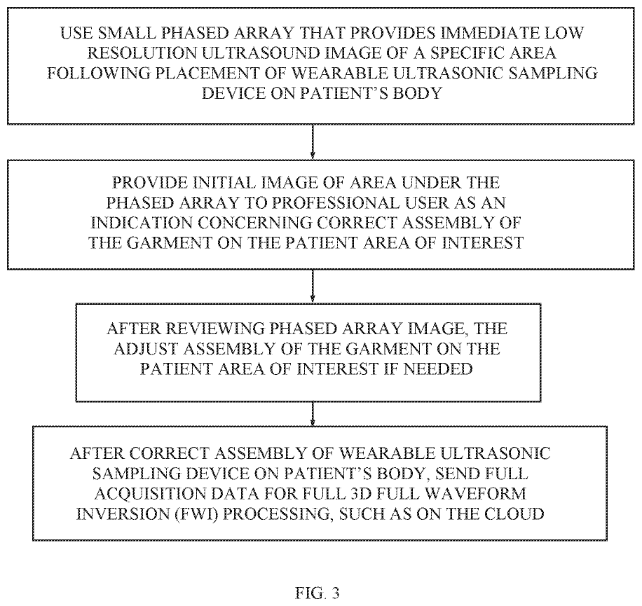

Reference is now made to FIG. 3. In one embodiment of the invention, a method is provided for evaluating patient US scan quality by a wearable ultrasound device. The method includes using a small phased array that provides an immediate (low resolution) ultrasound image of a specific area following placement of the wearable ultrasonic sampling device on the patient's body.

An initial image of the area under the phased array is provided to the professional user as an indication concerning the correct assembly of the garment on the patient area of interest. After reviewing the phased array image, the professional user may adjust the assembly of the garment on the patient area of interest if needed.

After correct assembly of the wearable ultrasonic sampling device on the patient's body, the professional user can send the full acquisition data for full 3d full waveform inversion (FWI) processing, such as on the cloud.

In accordance with an embodiment of the invention, a processor is provided for performing all the steps of the method, that is, an apparatus for generating optimized patient scan and enable professional user to evaluate the correct positioning of a wearable ultrasonic sampling device, which includes a small phased array located on a dedicated area on the wearable ultrasonic sampling device, a processing unit for analyzing the phased array signal, optionally a storage medium for computer readable images, an image visualization unit (e.g., display) for viewing the phased array image, and a signal receiving system for receiving input data regarding the quality scan and sending the full acquisition data to the cloud for full FWI algorithm processing.

Accordingly, this method can analyze specific transceiver element reflection patterns in specific key locations using a small phased array located on the garment and evaluate the garment position based on the initial reflection patterns on these specific key points even without direct imaging of the specific key points. This immediate reflection on the specific key point can indicate whether the transducer is located next to air, soft tissue or bone.

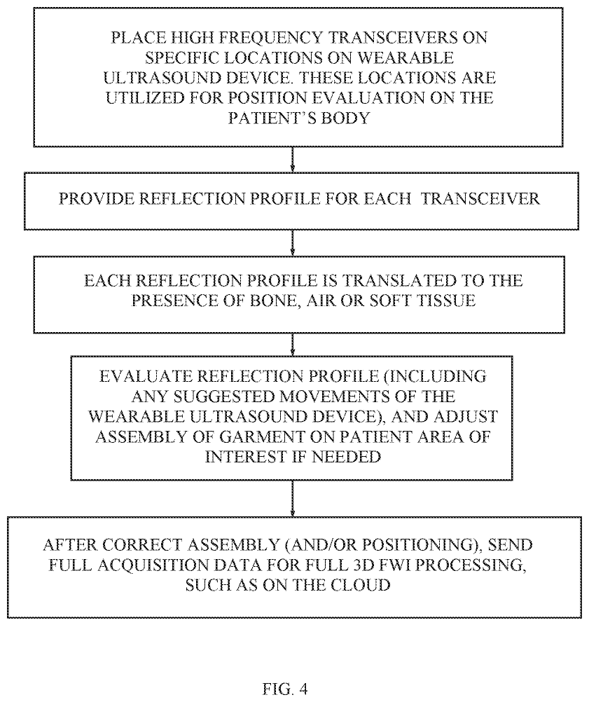

Reference is now made to FIG. 4. In another embodiment of the invention, a method is provided for evaluating patient scan quality by a wearable ultrasound device. The method includes placing high frequency transceivers on specific locations on the wearable ultrasound device, these locations being utilized for position evaluation on the patient's body. A reflection profile is provided for each of the transceivers. Each reflection profile is translated to the presence of bone, air or soft tissue.

The professional personnel may evaluate the reflection profile (including any suggested movements of the wearable ultrasound device), and adjust the assembly of the garment on the patient area of interest if needed.

After correct assembly (and/or positioning), the professional user may send the full acquisition data for full 3D FWI processing, such as on the cloud.

In accordance with an embodiment of the invention, a processor is provided for performing all the steps of this method, that is, an apparatus for generating optimized patient scan and enable professional user to evaluate the correct positioning of the wearable ultrasound device. The apparatus includes high frequency transceivers located on specific locations on the wearable ultrasound device. A processing unit receives the transceiver reflection profile and presents it to the professional user. A signal receiving system receives input data regarding the scan quality and sends the full acquisition data to the cloud for full FWI algorithm processing.

Accordingly, this method uses low resolution images to provide visual feedback to the patient and professional medical staff about the placement of the US sampling garment over the internal (yet invisible) organs. The feedback is provided at a fast turnaround time, yet at a resolution that enables the professional staff to determine that the garment is placed correctly. This is done within a delay that is small enough to be considered “real time”. The method allows the professional medical staff to adjust the garment (or transceiver location) before the heavy compute tasks are performed.

1. A method of evaluating patient scan quality by a wearable ultrasound device comprising:
a. assembling a garment comprising a wearable ultrasound device on a patient, and using said wearable ultrasound device to provide an ultrasonic image of a patient area of interest without full waveform inversion (FWI) processing;
b. providing an indication of positioning of said wearable ultrasound device on the patient area of interest;
c. following a review of said ultrasonic image, adjusting a position of said wearable ultrasound device if needed;
d. after correct positioning, sending full acquisition data acquired by said wearable ultrasound device for full 3D FWI processing.

2. The method according to claim 1, wherein providing said ultrasonic image of the patient area of interest is done by phased array imaging.

3. The method according to claim 1, wherein the full 3D FWI processing is done by a cloud based processor.

4. The method according to claim 1, wherein said ultrasonic image is used as the indication of positioning of said wearable ultrasound device on the patient area of interest.

5. The method according to claim 1, wherein said garment comprises transceivers located on specific locations on said garment, and the method comprises providing a reflection profile for each of said transceivers, each of said reflection profiles being translated to a presence of bone, air or soft tissue, and at least one of said reflection profiles is used as the indication of positioning of said wearable ultrasound device on the patient area of interest.

6. The method according to claim 1, wherein before said full 3D FWI processing, no inversion process is performed with said ultrasonic image.
